(19) United States
(12) Patent Application Publication
(10) Pub. No.: US 2009/0018631 A1
(43) Pub. Date: Jan. 15, 2009

(54) Surgical insertion apparatus

(76) Inventor: Elizabeth Ann Snoderly, Murrells Inlet, SC (US)

Correspondence Address: MCNAIR LAW FIRM, P.A., P.O. BOX 10827, GREENVILLE, SC, 29603-0827, US

(21) Appl. No.: 12/006,165

(22) Filed: Dec. 31, 2007

Related U.S. Application Data
(60) Provisional application No. 60/877,979, filed on Dec. 29, 2006.

(51) Int. Cl. A61N 1/05

(52) U.S. Cl. 607/117

(57) ABSTRACT

This invention is an apparatus for inserting and reinserting a medical device in a patient comprising a medical device such as a intrathecal catheter, electrical lead and lead wire, a sheath for being inserted into the patient using a hollow medical device so that once the sheath is inserting subcutaneously into the patient, the sheath receives the medical device and the medical device can be position in the patient; and, a separation portion carried by the sheath so that after the sheath receives the medical device. The sheath can be removed from around the medical device by using the separation portion so that the medical device can be inserted in the patient without having to remove the medical completely from the patient thus eliminating the requirement for a second incision.

### CROSS-REFERENCE TO RELATED APPLICATION

This patent application claims priority on U.S. Provisional Patent Application No. 60/877,979, filed Dec. 29, 2006.

### FIELD OF THE INVENTION

This invention is directed to a surgical tool and more specifically, to an internal medical device, including a spinal cord stimulation lead or intrathecal catheter, reinsertion tool.

### BACKGROUND OF THE INVENTION

In the treatment of some medical conditions, medical devices are inserted into a patient on a temporary, if not permanent, basis. For example, spinal cord stimulation (SCS) is a medical treatment procedure that uses an electrical current to treat chronic low back pain. It has been shown that electrical impulses applied to the spinal cord can assist with relieving pain in the back. In order to apply these electric impulses to the spinal cord, a medical device having spinal cord stimulation leads connected to an electrical source through lead wires are surgically inserted into the patient's back and placed in proximity to the spinal cord. Specifically, leads are connected to a small electrical pulse generator that can be implanted in the patient, preferably on the hip or in the back that transmits electrical pulses to the spinal cord through lead wires. These pulses interfere with the nerve impulses responsible for chronic low back pain or leg pain and numbness and relieve the pain of the patient.

When these medical devices are inserted, there are several instances when the medical device has to be reinserted. For example, in SCS, one of the most common complications is lead migration. This condition exists when the leads that were placed in the patient move away from the location where they were initially implanted. Further, studies have shown that three out of sixty patients, or five percent, required lead replacement within thirty days of the procedure. Eleven out of sixty patients required lead repositioning during follow-up examinations after the initial thirty days. Therefore, twenty-three percent of the patients required additional surgery specifically for lead replacement.

When inserting a particular medical device which is generally thin, a hollow, fairly rigid device is first placed in the patient to provide a path or guide for the thin medical device to travel. For example, an epidural needle, commonly known as a Touhy needle, can be used. Although many hollow medical devices can be used for placement of the thin medical device within patients, the disclosure herein will refer to such hollow device, including epidural device, as Touhy needles.

When applying SCS, a Touhy needle is used during the initial insertion of the leads. A Touhy needle is generally a hollow needle having an angled tip. The tip can be slightly curved. A solid stylet can be positioned inside the Touhy needle and substantially fill the hollow space of the Touhy needle to assist with its insertion into a patient. Once the skin has been traversed by the Touhy needle, the solid stylet can be removed from within the hollow needle and the lead can be placed in the Touhy needle for placement within the patient and in proximity to the spine. To remove the Touhy needle after the lead has been inserted; the Touhy needle slides the entire length of the lead and lead wire and is removed. The lead wire is then inserted under the skin and the connector end is placed generally in the hip area where the pulse generator is located. The lead wires are then connected to the pulse generator. Therefore, during the initial insertion of the leads and the lead wires, the Touhy needle can be removed by sliding the Touhy needle along the lead wires since the lead wires are not yet placed under the skin of the patient and are not connected to the pulse generator.

When repairing lead migration, the leads and lead wires are already placed under the skin of the patient. Traditionally, to repair lead migration, a first incision is made at the lead location and a second incision made at or near the pulse generator so that the lead and the lead wires can be disconnected from the pulse generator and the lead wires pulled through the first incision. Through the first incision, a Touhy needle is reinserted in the patient and used to replace the leads. The lead wires are then pulled out of the patient after being detached from the pulse generator so that the Touhy needle can slide along the lead wires to be removed. The lead wires are then replaced under the skin of the patient and reattached to the pulse generator through the second incision. Therefore, the second incision is necessary to detach the lead wires from the pulse generator and to allow the Touhy needle to be removed once it has been used to reinsert the leads which have migrated.

The same problem exists when other medical devices need to be reinserted, but both ends are inserted into the patient. Again, use of the Touhy needle is impossible without removing the medical device from the patient so that the medical device allows the Touhy needle to completely slide off the medical device. Another example of a medical device that has the same problem with reinsertion is an intrathecal pump which can be used to deliver drugs to a delivery site. An intrathecal pump is a specialized device, which delivers concentrated amounts of medication into spinal cord area through an intrathecal catheter. This device delivers concentrated amounts of medication into spinal cord area allowing the patient to decrease or eliminate the need for oral medications and can be used for treating pain. It delivers medication around the clock, thus eliminating or minimizing pain or other symptoms. Similar to the SCS treatment, an intrathecal catheter is inserted in the midline at the lower back and the pump is placed on the side of the abdomen.

Just as with the electrical leads, the intrathecal catheter is inserted into the patient at a specific drug delivery site and has both the distal end inserted through an incision and the drug pump in another position. The intrathecal catheter can migrate from the desired drug delivery site and therefore needs to be repositioned. In fact, catheter migration is a frequently reported complication of intrathecal drug delivery systems.

It would be advantageous to have a surgical instrument that would provide a means for reinserting medical devices such as SCS leads and intrathecal catheters through the first incision without having to make a second incision. It would be advantageous to have a medical device that would allow reinsertion of leads, catheters, or other type of subcutaneous medical devices using Touhy needles without having to completely remove the medical device to remove the Touhy needle.

### SUMMARY OF THE INVENTION

This invention is an apparatus for inserting and reinserting a medical device in a patient comprising a medical device such as a intrathecal catheter, electrical lead and lead wire, a sheath for being inserted into the patient using a hollow medical device so that once the sheath is inserting subcutaneously into the patient, the sheath receives the medical device and the medical device can be position in the patient; and, a separation portion carried by the sheath so that after the sheath receives the medical device. The sheath can be removed from around the medical device by using the separation portion so that the medical device can be inserted in the patient without having to remove the medical completely from the patient thus eliminating the requirement for a second incision.

The separation portion of the sheath can be one of a perforation strip, a plurality of perforation strips and a separation strip. The tear portion can also include a hook edge carried by the sheath and a trough edge carried by the sheath for receiving the hook edge to define a sheath interior space for receiving the medical device. The separation portion can include a first edge carried by the sheath positioned to overlap a second edge carried by the sheath to define a sheath interior space for receiving the medical device.

### DESCRIPTION OF THE DRAWINGS

The invention will be more readily understood by reviewing the following drawings and the specification.

FIG. 1 is a rear view of a patient;

FIGS. 2A-2E are a cross sections showing a medical device inserted;

FIG. 3 is an internal view of a patient showing a medical device inserted;

FIGS. 4A-4B are portions of an internal view of a patient;

FIGS. 5A-5G are cross sections of the patient showing the invention;

FIGS. 6A-6J are perspective views of the invention; and,

FIGS. 7A-7C are cross sections of the patient showing the invention.

### DESCRIPTION OF THE INVENTION

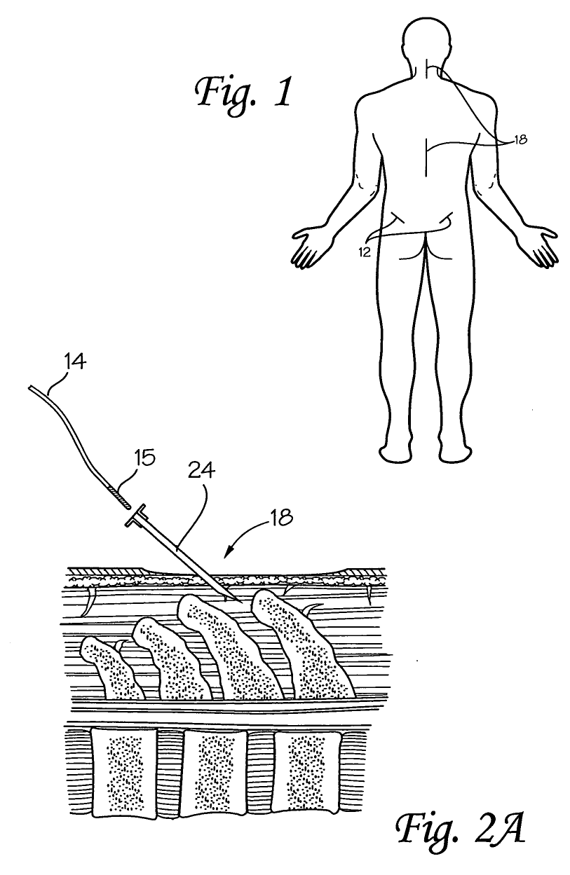

Referring now to FIG. 1, the process of inserting a pulse generator and leads is shown. It is to be understood that this invention is not limited to SCS leads, but can apply to other medical devices having characteristics of SCS leads.

The initial SCS procedure begins by making an incision 18 and incision 12 in the patient. The incision 18 is for insertion of one or more leads to be placed in proximity to the spinal column. This incision can be located at various positions along the spine. Insertion 12 is for inserting a pulse generator into the patient and for connecting the lead wires to the pulse generator.

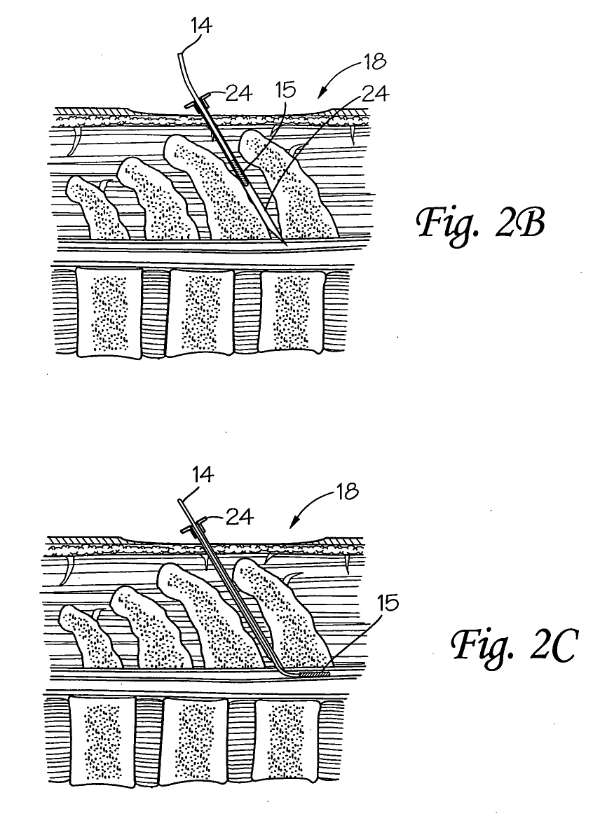

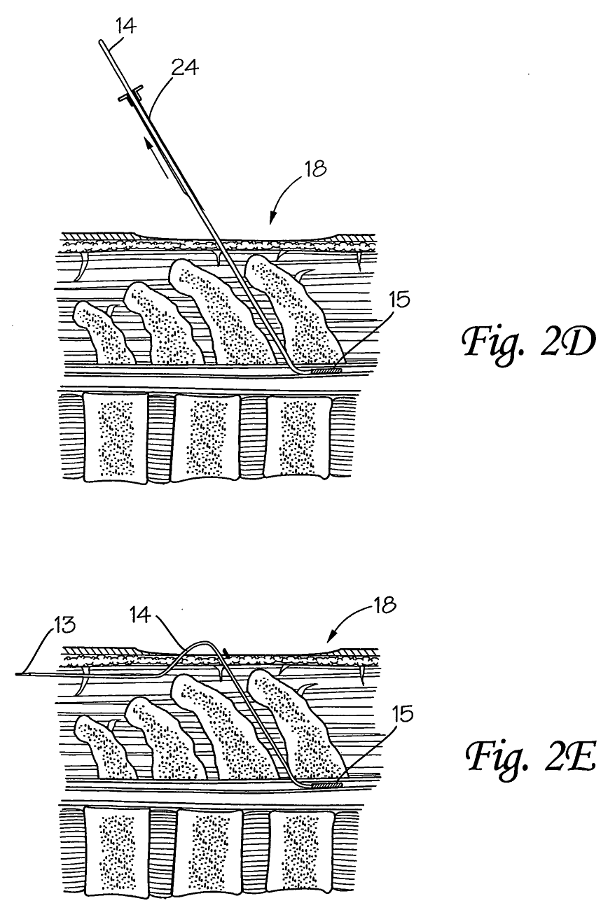

Referring now to FIG. 2A the initial insertion of the lead is shown. Incision 18 is shown with a Touhy needle 24 inserted through incision 18. Once the Touhy needle is inserted, lead 15, connected to lead wire 14, can be placed through Touhy needle 24. In FIG. 2B, lead 15 attached to lead wire 14 is inserted into the Touhy needle so that it can be placed adjacent to spine 16 of the patient. In FIG. 2C, the lead is shown extending from the distal end of the Touhy needle and adjacent to spine 16. Lead wire 14 is extending from the other end of the Touhy needle. At this point, the lead is positioned in its location desirable by the medical practitioner and the Touhy needle needs to be removed. In FIG. 2D, the Touhy needle is removed from out of incision 18 along lead wire 14. In FIG. 2E, with the Touhy needle completely removed, lead wire 14 is placed in incision 18 and inserted under the skin until the connection in 13 is in proximity to pulse generator 10. Since the lead wires have not been inserted into the patient, the Touhy needle can slide along the lead wire and be removed prior to the lead wire being placed in the patient.

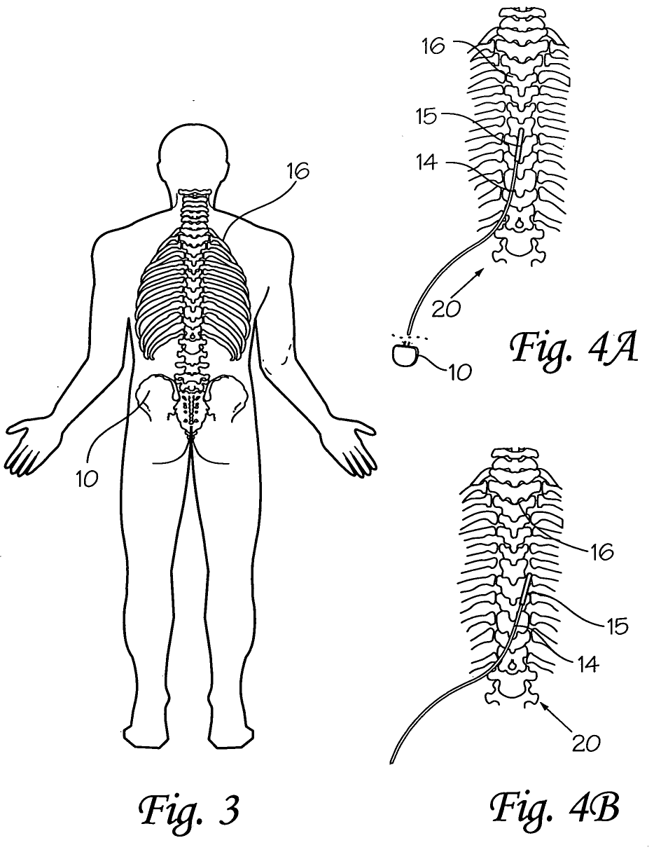

Referring now to FIG. 3, pulse generator is shown inserted through incision 12 in the hip of the patient. Lead wires 14 are connected to the pulse generator and the leads are in proximity to spine 16. The incisions are closed and the operation is complete.

Referring now to FIG. 4A, lead 15 is shown properly placed adjacent to spine 16 with lead wire 14 traveling to the pulse generator 10. In FIG. 4B, lead 15 has migrated to the right of spinal column 16 and is not properly placed for the patient to rely on the full benefit of SCS. Sometimes the lead can even migrate out of the epidural space. Therefore, the lead needs to be repositioned over the spine due to this migration. It can be seen that once the lead is in position, the Touhy needle cannot travel along the lead wire since it is under the skin and attached to the pulse generator. As such, a Touhy needle cannot serve the purpose of re-inserting a lead once it is migrated.

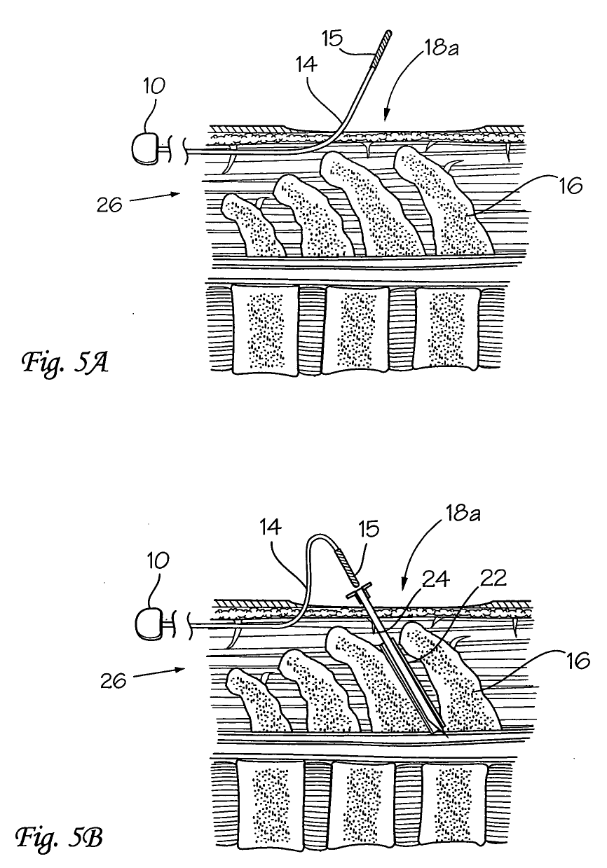

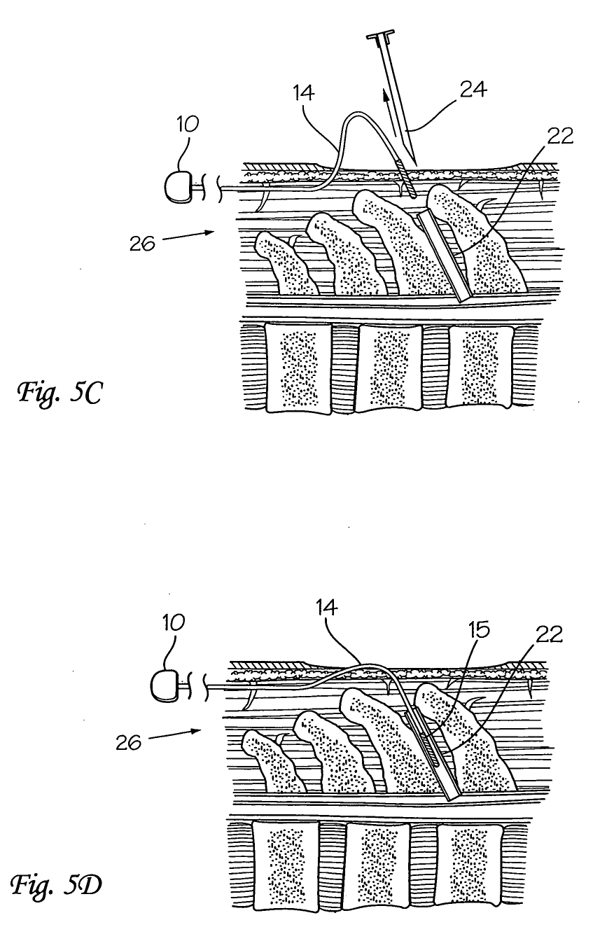

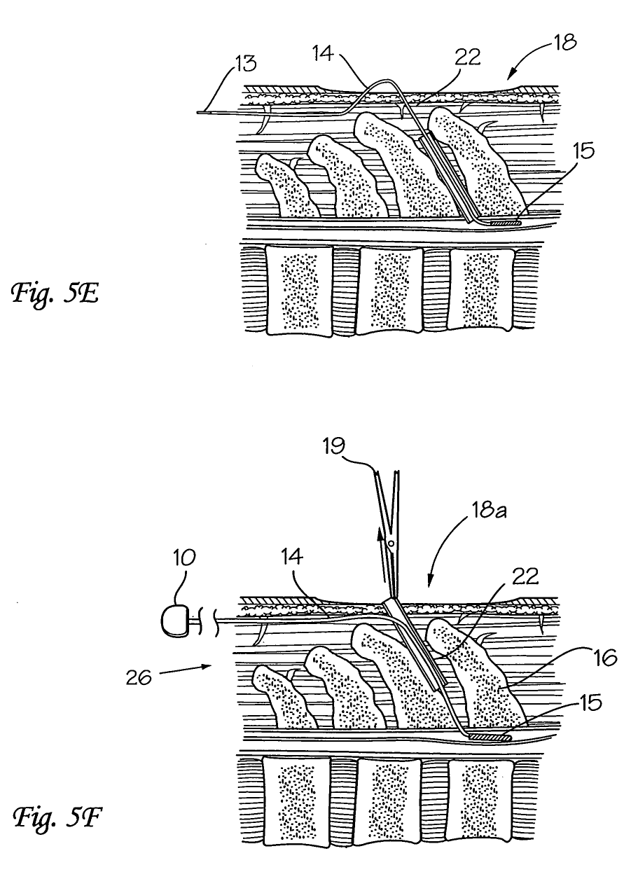

Referring now to FIG. 5A, another incision 18a is made in the patient. Lead 15 can be pulled through incision 18a to be re-inserted in the proper location adjacent to spine 16. However, since lead 14 travels under the skin and is connected to pulse generator 10, the use of the Touhy needle would require that lead wire 14 be pulled completely out of incision 18a to allow the Touhy needle, once used, to slide all the way down lead 14. This would require lead 14 to be detached from pulse generator or extension 10 and would also require a second incision proximate to the lead pulse generator to allow for the re-attachment of the lead wire to the pulse generator.

Referring to FIG. 5B, the Touhy needle is used to insert the sheath into the patient so that a distal end is in the epidural space of the patient. The Touhy needle is then removed from the patient leaving the sheath in place as shown in FIG. 5C. It should be noted that the sheath can be positioned so that the end receiving the medical device can be underneath or above the skin of the patient. Once the Touhy needle has been removed, the lead can be inserted into the sheath so that the distal end of the lead enters the epidural space of the patient through the opening in the sheath as shown in FIG. 5D. The sheath is then torn away from around the lead and the lead wire and can be removed from the patient a shown in FIG. 5E. The sheath can also be removed with the assistance of removal tool 19 shown in FIG. 5F. Once the sheath has been removed, the lead wire can be placed in the incision of the patient and incision 18a can be closed.

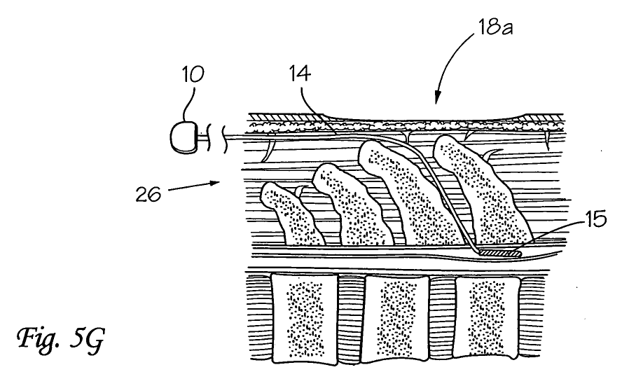

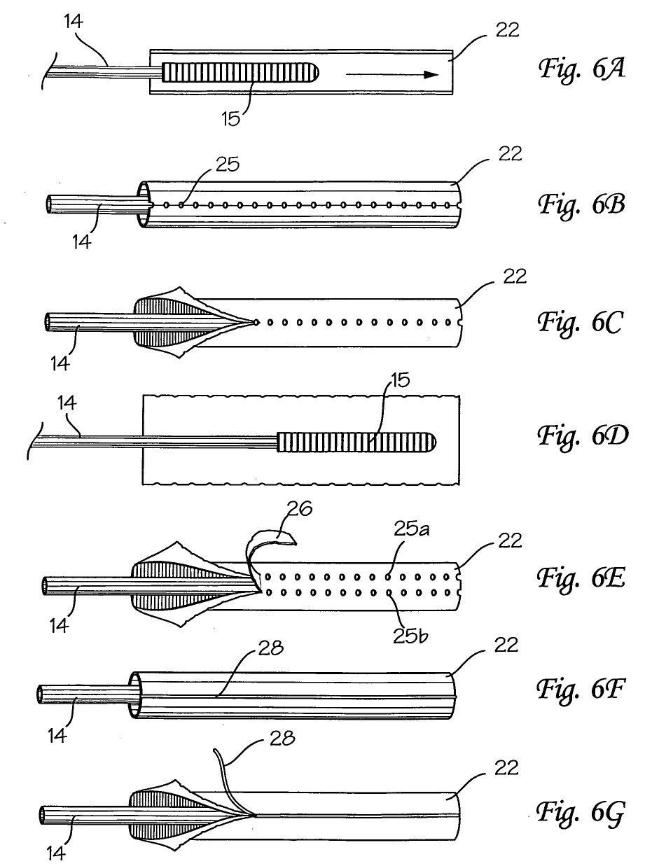

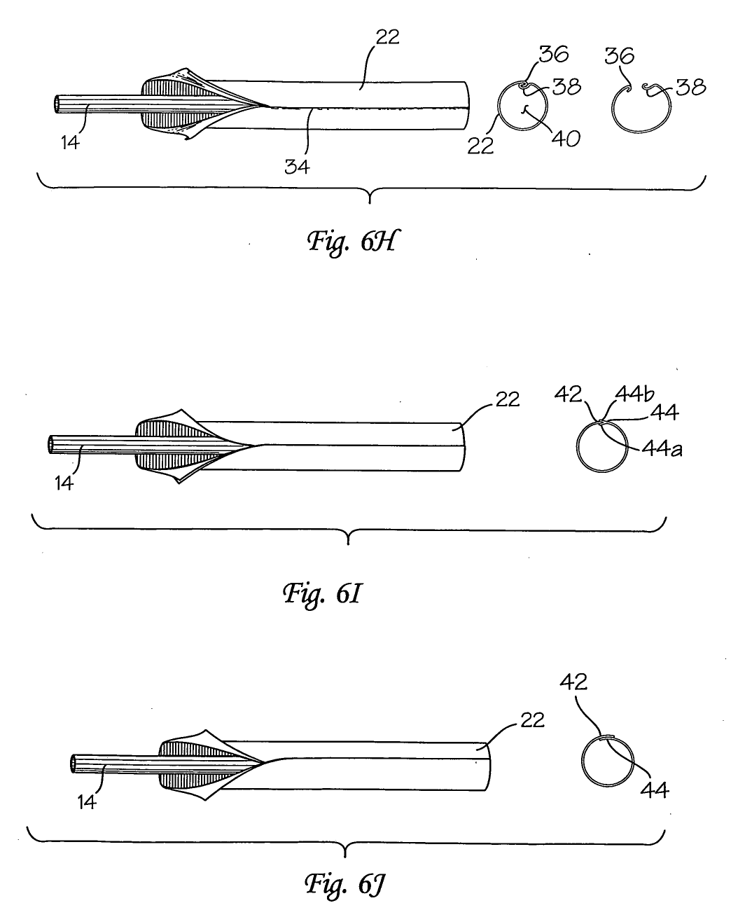

Referring now to FIGS. 6A-6I, several embodiments are shown of sheath 22 which can receive a medical device such as lead 15 and lead wire 14 to insert or reinsert the medical device back in the patient. Sheath 22 can have a series of perforations shown as 25 (FIG. 6B) to allow the sheath to peel away from the medical device. Therefore, the sheath can be removed from around the medical device without having to create a second incision since the sheath need not travel down the medical device to be removed. In another embodiment, sheath 24 can have a plurality of rows of perforations shown as 25a and 25b (FIG. 6E) generally to allow a strip 26 to be peeled off the sheath and again allow the sheath to peel away from the medical device. In another embodiment, sheath 24 can have a tear strip 28 (FIG. 6F) which can be removed from sheath 24 and again allow the sheath to peel away from the medical device.

Referring to FIG. 6H, another embodiment of the sheath is illustrated. Sheath 22 has separation portion 34. Separation portion can also be perforations or a tear strip. Hook edge 36 is received into trough edge 38 thereby defining a sheath interior space 40 to receive a medical device. When hook edge 36 is disengaged from trough edge 38, the sheath can be removed from around the sheath.

Referring to FIG. 6I, another embodiment of the sheath is illustrated. First, edge 42 can be received by second edge 44 to define sheath interior space 40. Second edge 44 includes first leg 44a and second leg 44b. When first edge 42 is separated from second edge 44, the sheath can be removed from the medical device. In another embodiment, first edge 42 can overlap second edge 44 to define the sheath interior space as shown in FIG. 6J. In this embodiment, leg 44 is not included.

Therefore, the medical device can be repositioned without the need for a second incision so that less risk of infection and other hazards of surgery are reduced when inserting or reinserting a medical device. It should be noted that any of the embodiments shown in FIGS. 6A-6G can be used to peel the sheath away from the medical device, therefore allowing only a single incision.

It should be understood that SCS has been used to explain the interaction of the lead, Touhy and sheath. For example, this invention can also be used to reinsert intrathecal catheters. Further, any medical device having similar physical characteristics as SCS leads and intrathecal catheters can be reinserted with this invention.

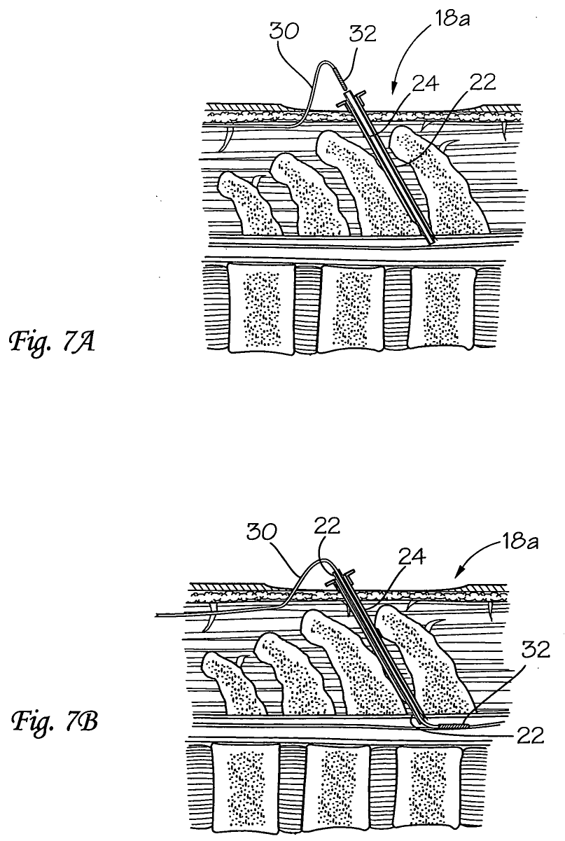

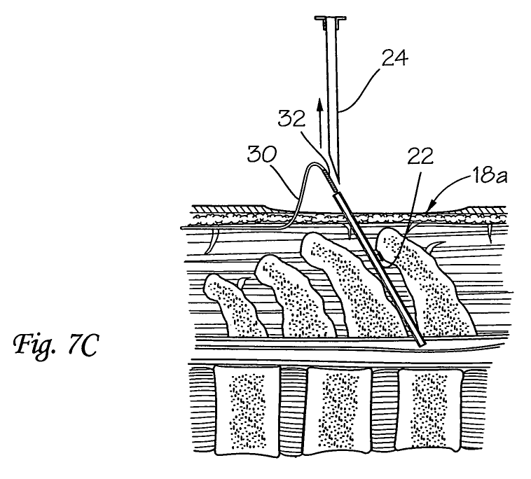

Referring now to FIG. 7A, another embodiment of this invention is shown. Medical device 30 needs to be inserted into the patient through incision 18a. However, medical device 30 has one and that is already inserted in the patient. Further, it would be advantageous to use a Touhy needle to insert the medical device. As previously described, if the Touhy needle is inserted into the patient and the medical device placed in the Touhy needle, the Touhy needle cannot be removed. This problem is illustrated by FIG. 7B. In FIG. 7B, the distal end 32 of medical device 30 is placed through Touhy needle 24 and inserted into the patient. The Touhy needle cannot be removed from the patient. Therefore, the sheath as described herein can be used to provide an insertion tool that is not “trapped” in the patient once distal end 32 is in the patient.

Referring now to FIG. 7C, Touhy needle 24 is shown removed while sheath 22 remains in the patient. This allows medical device 30 to be placed in the patient, through sheath 22 and allows the sheath to be removed as described herein. It should be noted that the sheath can be semi-rigid or have little structural support since structural support is being provided by the Touhy needle during insertion.

This invention may also be used for repositioning intrathecal catheters in the same manner as described herein.

While a preferred embodiment of the invention has been described using specific terms, such description is for illustrative purposes only, and it is to be understood that changes and variations may be made without departing from the spirit or scope of the following claims.

What is claimed is:

1. An apparatus for positioning an electrical lead in a patient comprising:
an electrical pulse generator having a lead wire and an electrical lead connected to said lead wire wherein a portion of said lead wire is subcutaneous in the patient;
a sheath for being inserted into the patient using a hollow medical device so that once said sheath is inserting into the patient said sheath receives said electrical lead so that said electrical lead can be position in the patient; and,
a separation portion carried by said sheath so that after said sheath receives said electrical lead and said electrical lead is positioned in the patient, said sheath can be removed from around said lead wire by using said separation portion so that said electrical lead can be inserted in the patient without having to remove the lead wire completely from the patient thus eliminating the requirement for a second incision.

2. The apparatus of claim 1 wherein said separation portion of said sheath is one of a perforation strip, a plurality of perforation strips, a tear away strip and opposing sheath edges.

3. The apparatus of claim 1 including a sheath interior space defined in said sheath for receiving a hollow medical device to position said sheath in the patient.

4. The apparatus of claim 3 wherein said hollow medical device is a Touhy needle.

5. The apparatus of claim 1 wherein said separation portion includes a hook edge carried by said sheath for receiving said hook edge to define a sheath interior space for receiving said electrical lead and said lead wire and for allowing said sheath to be removed from around said lead wire.

6. The apparatus of claim 1 wherein said separation portion includes a first edge carried by said sheath positioned to overlap a second edge carried by said sheath to define a sheath interior space for receiving said electrical lead and lead wire and for allowing said sheath to be removed from around said lead wire.

7. An apparatus for repositioning an intrathecal catheter in a patient comprising:
a pump having an intrathecal catheter connected to said pump;
a sheath for being inserted into the patient using a hollow medical device so that once said sheath is inserting subcutaneously into the patient, said sheath receives said intrathecal catheter said intrathecal catheter can be position in the patient; and,
a separation portion carried by said sheath so that after said sheath receives said intrathecal catheter and said intrathecal catheter is positioned in the patient, said sheath can be removed from around said intrathecal catheter by using said separation portion so that said intrathecal catheter can be inserted in the patient without having to remove the intrathecal catheter completely from the patient thus eliminating the requirement for a second incision.

8. The apparatus of claim 7 wherein said separation portion of said sheath is one of a perforation strip, a plurality of perforation strips, a tear away strip and opposing sheath edges.

9. The apparatus of claim 7 including a sheath interior space defined in said sheath for receiving a hollow medical device to position said sheath in the patient.

10. The apparatus of claim 9 wherein said hollow medical device is a Touhy needle.

11. The apparatus of claim 7 wherein said separation portion includes:
a hook edge carried by said sheath; and,
a trough edge carried by said sheath for receiving said hook edge to define a sheath interior space for receiving said intrathecal catheter and for allowing said sheath to be removed from around said intrathecal catheter.

12. The apparatus of claim 7 wherein said separation portion includes a first edge carried by said sheath positioned to overlap a second edge carried by said sheath to define a sheath interior space for receiving said intrathecal catheter and for allowing said sheath to be removed from around said intrathecal interior.

13. An apparatus for inserting a medical device in a patient comprising:
a medical device;
a sheath for insertion into the patient and for receiving said medical device to insert said medical device in the patient; and,
a separation portion carried by said sheath so that said sheath can be removed via said separation portion from around said medical device to allow said medical device to be inserted in the patient.

14. The apparatus of claim 13 wherein said medical device is one of an electrical lead and an intrathecal catheter.

15. The apparatus of claim 13 including a sheath interior space defined in said sheath for receiving a hollow medical device to position said sheath in the patient.

16. The apparatus of claim 15 wherein said hollow medical device is a Touhy needle.

17. The apparatus of claim 13 wherein said separation portion of said sheath is one of a perforation strip, a plurality of perforation strips, a tear away strip and opposing sheath edges.

18. The apparatus of claim 13 wherein said separation portion includes:
a hook edge carried by said sheath; and,
a trough edge carried by said sheath for receiving said hook edge to define a sheath interior space for receiving said medical device and for allowing said sheath to be removed from around said medical device.

19. The apparatus of claim 13 wherein said separation portion includes a first edge carried by said sheath positioned to overlap a second edge carried by said sheath to define a sheath interior space for receiving said medical device and allowing said sheath to be removed from around said medical device.

20. The apparatus of claim 19 wherein said second edge includes a first leg and a second leg so that said first edge overlaps said first leg and said second leg to define said sheath interior space.
